(19) United States
(12) Patent Application Publication
(10) Pub. No.: US 2005/0025769 A1
(43) Pub. Date: Feb. 3, 2005

(54) Method of suppressing ongoing acute allograft rejection

(75) Inventors: Masakazu Kobayashi, Glenview, IL (US); Hongsi Jiang, Wilmette, IL (US); Xiu-Fen Yang, West Haven, CT (US)

Correspondence Address: LEYDIG VOIT & MAYER, LTD, 700 THIRTEENTH ST. NW, SUITE 300, WASHINGTON, DC, 20005-3960, US

(73) Assignee: Fujisawa Pharmaceutical Company, Ltd., Osaka-shi (JP)

(21) Appl. No.: 10/931,997

(22) Filed: Sep. 2, 2004

Related U.S. Application Data
(63) Continuation of application No. 09/889,837, filed on Oct. 29, 2001.
(60) Provisional application No. 60/116,845, filed on Jan. 22, 1999.

(51) Int. Cl. A61K 39/395; A61K 38/13

(52) U.S. Cl. 424/145.1; 514/11

(57) ABSTRACT

The present invention provides a method of suppressing ongoing acute allograft rejection. In one embodiment, the method comprises administering to a host experiencing ongoing acute allograft rejection an IL10 inhibitor and an IL2 inhibitor in amounts effective to rescue the allograft from ongoing acute rejection. In another embodiment, the method comprises administering to a host experiencing ongoing acute allograft rejection, which is due to insufficient immunosuppression by an IL2 inhibitor, an IL10 inhibitor in an amount effective to rescue the allograft from ongoing acute rejection.

### CROSS REFERENCE TO RELATED PATENT APPLICATIONS

This patent application is a continuation of application Ser. No. 09/889,837, filed Jul. 20, 2001, which is a 371 of International Application No. PCT/US00/01553, filed Jan. 21, 2000, which claims the benefit of provisional application Ser. No. 60/116,845, filed Jan. 22, 1999.

### TECHNICAL FIELD OF INVENTION

The present invention relates to a method of suppressing ongoing acute allograft rejection.

### BACKGROUND OF THE INVENTION

Acute organ or tissue allograft rejection remains a formidable clinical problem. For example, acute rejection is the most serious and frequent complication following kidney transplantation (Suthanthiran et al., Renal Transpl., 78, 77-94 (1998)). Acute rejection occurs when the recipient immune system recognizes the transplanted graft as foreign and initiates an immune response to destroy it. Acute rejection is a cell-mediated immune response orchestrated primarily by CD4⁺ T-lymphocytes (helper T cells) which recognize donor (i.e., graft) major histocompatibility complex (MHC) epitopes and secrete cytokines stimulating the production of CD8⁺ T-lymphocytes (cytotoxic T cells). The activities of the cytotoxic T cells, in turn, cause injury to the graft.

At least two subpopulations of CD4⁺ T cells, which are differentiated on the basis of the cytokines that they produce, regulate graft rejection. Helper T1 cells (Th1) produce proinflammatory cytokines, including interleukin-2 (IL2), interferon-γ (IFNγ), tumor necrosis factor-β (TNFβ) and lymphotoxin, which stimulate the cell-mediated immune response. In contrast, helper T2 cells (Th2) secrete interleukin-4 (IL4), interleukin-5 (IL5), interleukin-6 (IL-6), interleukin-10 (IL10) and interleukin-13 (IL13), among others, which are involved in the stimulation of humoral and allergic responses (Mossman et al., Ann. Rev. Immunol., 7, 145-173 (1989); Seder et al., Ann. Rev. Immunol., 12, 635-673 (1994); (Paul et al., Cell, 76, 241-251 (1994); Ferrara et al., Transfusion Med. Rev., 12, 1-17 (1998)). In addition, the Th2-associated cytokines, such as IL4 and IL10, have been shown to inhibit the activation of Th1 cells and to constrain the antigen-presenting function of macrophages (Moore et al., Ann. Rev. Immunol., 11, 165-190 (1993)).

In vitro and in vivo transplantation models have demonstrated that the activation of Th1 cells results in allograft rejection. The activation of Th1 cells reportedly facilitates the function of cytotoxic activities, such as those involving IL2 and IFN (Novelli et al., J. Immunol., 147, 1445-1450 (1991); Landolfo et al., Science 229, 176-180 (1985)).

Similarly, the Th1-associated cytokines have been demonstrated to be involved in many autoimmune diseases. For example, patients with rheumatoid arthritis have predominantly Th1 cells in synovial tissue (Simon et al., PNAS, 91, 8562-8566 (1994)) and experimental autoimmune encephalomyelitis (EAE) has been shown to be induced by Th1 cells (Kuchroo et al., J. Immunol., 151, 4371-4381 (1993)). Furthermore, Th1-associated cytokines have been shown to modulate the activation and function of endothelial cells, keratinocytes and hepatocytes as well as immune cells.

The exact role of Th2 cells and their associated cytokines in acute rejection and autoimmune disease remains very controversial. The silencing of the Th1-type cytokine program and the inhibition of the antigen-presenting function of macrophages by Th2-associated cytokines suggest that Th2 cytokines function as anti-inflammatory agents and immunosuppressors. Thus, elevated levels of Th2 cytokines would be expected to play a role in enhancing graft survival and inhibiting the autoimmune response.

However, the ability of Th2-associated cytokines to up-regulate IL2-independent T cell activation, initiate B cell function and enhance CD8⁺ T cell and NK cell cytotoxicity suggests that Th2-associated cytokines also may function as immunostimulators. In support of this, recent experimental and clinical studies have shown that Th2-type cytokine mRNAs are up-regulated in grafts undergoing acute rejection (Fujimura et al., Transpl. Proc., 30, 1023-1026 (1998); Nagano et al., Transpl., 63, 1101 (1997); Suthanthiran, Med. Sci., 313, 264 (1997), Great et al., Transpl., 62, 910 (1996); Orosz et al., Transpl., 63, 1109 (1997)). In addition, recent investigations have indicated that TH2 responses participate in allergic disease, with IL4 detected in the T cells from the bronchi of patients with asthma and in the T cells from the blood and lesions of atopic dermatitis (Ohmen et al., J. Immunol. 154: 1956-1963 (1995)). Monocytes, rather than T-cells, appear to be the major source of IL10 in atopic dermatitis.

The role of the Th2-associated cytokine IL10 in graft rejection and autoimmune responses is particularly controversial. IL10 was first described as a cytokine synthesis inhibitor factor (CSIF), which was produced by Th2 cells and which inhibited production of cytokines, such as INFγ, IL1α, IL1β, IL2 and TNFα, by Th1 cells (Fiorentino et al., J. Immunol., 146, 3444-3451 (1991); Moore et al., Science 248, 1230-1252 (1990)). In addition to inhibiting the production of proinflammatory cytokines, IL10 has been shown to inhibit antigen-specific Th1 cell proliferation by reducing the antigen-presenting capacity of monocytes through the down-regulation of class II MHC antigen expression on these cells (Trowbridge et al., J Expt. Med., 154, 1517-1524 (1981); Lambert et al., Cell. Immunol., 120, 401-418 (1981); Mossman et al., Immunol. Today, 12, A49-A53, (1991)). Others have shown that IL10 inhibits not only class II MHC expression but also B7 and Intracellular Adhesion Molecule-1 (ICAM-1) expression on human monocytes (Abramowicz, WO 94/17773). B7 and ICAM-1 are “accessory molecules” expressed on the membranes of antigen-presenting cells (APC) which interact with their counterpart receptors (CD28 and LFA-1, respectively) on the surfaces of the T cells and which aid in the co-stimulation of T cells. Therefore, it has been hypothesized that IL10 inhibits T cell activation by interfering with both the MHC II/T cell receptor interaction and the B7/CD28 and ICAM-1/LFA-1 interactions (Abramowicz, WO 94/17773).

The finding that IL10 has a strong inhibitory effect on the activation of Th1 cells and the production of proinflammatory cytokines led to the hypothesis that IL10 is a powerful immunosuppressant of cell-mediated immune responses, such as allograft rejection (Lang et al., Transpl. Proc. 27, 1146-1147 (1995)). Thus, many have suggested the use of IL10 for suppressing graft rejection following organ transplantation (Warrick, WO 97/05896; Abramowicz, WO 94/17773). Others have suggested the use of IL10 to treat acute and chronic inflammatory conditions as well as autoimmune diseases (Lauener et al., WO 96/32418; Tadmori, WO 97/05896). However, recent reports have suggested that IL10 also may be an immunostimulatory factor. For example, IL10 has been shown to support dendritic cell-induced CD8⁺ T cell differentiation, augment IL2-supported CD8⁺ T cell proliferation, and enhance alloantibody-dependent cellular cytotoxicity (Macatonia et al., J. Immunol., 150, 3755 (1993); Chen et al., J. Immunol., 147, 528-534 (1991); te Velde et al., J. Immunol., 149, 4048 (1992); Go et al., J. Exp. Med., 172, 1625-1631 (1990)). Further, IL10 has recently been shown to be up-regulated in allografts undergoing acute graft rejection, suggesting that IL10 may function as an accelerating factor in acute allograft rejection. (Fujimura et al., Transpl. Proc., 30, 1023-1026 (1998); Suthanthiran, Med. Sci., 313, 264 (1997); Orosz et al., Transpl., 63, 1109 (1997)).

Cyclosporin A (CsA), a cyclic peptide produced by the fungus Tolypocladium inflatum Gams and other fungi imperfecti, is widely used as an immunosuppressant for the prevention of graft rejection in transplant recipients. For example, CsA has been successful in prolonging the survival of allogeneic transplants involving skin, heart, kidney, pancreas, bone marrow, small intestine and lung, as well as suppressing graft-versus-host disease and delayed-type hypersensitivity. CsA has been shown to interfere with the activation of T lymphocytes and to inhibit the transcription of several cytokines, including IL2, IFNγ, and IL4 genes during progression from the G₀ to G₁ phase of the cell cycle (Flamand et al., J. Immunol., 144, 2875- (1990); Granelli-Piperno, J. Exp. Med., 171, 533 (1990); Sigal et al., Ann. Rev. Immunol., 10, 519 (1992); Tocci et al., J. Immunol., 143, 718-726 (1989)). Furthermore, CsA has been shown to suppress the expression of the interleukin-2 receptor and the release of IL2 (Kino et al., J. Antibiot., 40, 1256-1265 (1987); Tocci et al., J. Immunol., 143, 718-726 (1989)) and to interfere with the activation of T cells (Tocci et al. (1989), supra).

The inhibitory effect of CsA is produced as a result of its binding to its intracellular binding protein cyclophilin and complexing with calcineurin, thereby inactivating calcineurin and blocking early activation of cytokines of which IL2 gene transcription and IL2 receptor expression have been intensively investigated (O'Keefe et al., Nature, 357, 692 (1992); Clipstone et al., Nature, 357, 695 (1992)). Calcineurin, a Ca²⁺-calmodulin-dependent protein phosphatase, is required for the functional activation of several DNA-binding proteins that bind to and participate in the transcriptional activation of the IL2 gene (Clipstone et al., Nature, 357, 695(1992); Schreiber, Cell, 70, 365 (1990); Martin et al., J. Immunol., 154, 922-927 (1995); Jain, et al., Nature, 365, 352-355 (1993)). It is thought that calcineurin may dephosphorylate a cytoplasmic T cell transcription factor, such as NF-AT or AP-1, thereby inducing the nuclear translocation of such a factor, allowing activation of the IL2 gene (Morris, Transpl. Proc., 26, 3272-3275 (1994); (Jain et al., Nature, 356, 801-804 (1992)).

Another widely used immunosuppressant is a macrolide antibiotic produced by Streptomyces tsukubaensis known as Tacrolimus or FK506. Despite their structural differences, tacrolimus inhibits T-cell activation by mechanisms that are similar to those of CsA (Thompson, Immunol. Today, 10, 6 (1989); Peters et al., Drugs, 46, 746-794 (1993); Ochiai et al., Transpl., 44, 729-733 (1987); Tocci et al., supra (1989)). Like CsA, tacrolimus binds to its corresponding cellular binding protein (FKBP), forming a complex which binds to and inhibits the activity of calcineurin, resulting in the inhibition of key signal transduction events in CD4⁺ Th cells that lead to the transcription of IL2 and other cytokine genes (Sigal et al., Ann. Rev. Immunol., 10, 519 (1992); Tocci et al., J. Immunol., 143, 718 (1989)). Notably, tacrolimus has been found to be 10 to 100 times more potent than CsA in vitro in inhibiting T-cell proliferation, IL2 production, and the production of other growth-promoting cytokines and 10 times more potent in vivo in suppressing T-cell-dependent antibody production, graft vs. host reactivity, and delayed-type sensitivity reactions (Morris et al., Transpl. Proc., 26, 3272 (1994)).

Clinical and experimental trials have shown that tacrolimus offers a promising alternative to CsA as a primary immunosuppressant for the prevention of acute allograft rejection (Ochiai et al., Transpl. Proc., 19, 1284 (1987); Ochiai et al., Transpl., 44, 729-733 (1987); Murase et al., Transpl., 50, 186-189 (1990); Todo et al., Transpl. Proc., 19, 64-67 (1987); Todo et al., Surgery, 104, 239-249 (1988); Todo et al., Ann. Surg., 212, 295-305 (1990)). tacrolimus has been tested in a wide variety of animal transplantation models, including skin, heart, liver, kidney, and small bowel allograft transplantation (Kino et al., J. Antibiot., 40, 1256 (1987); Sawada et al., J. Immunol., 139, 1797 (1987); Tocci et al., J. Immunol., 143, 718 (1989); Sigal et al., Ann. Rev. Immunol., 10, 519 (1992) Starzl et al., Lancet, 2, 1000-1004 (1989); Starzl et al., JAMA, 264, 63 (1990); Armitage et al., Transpl., 53, 164 (1991); Todo et al., Transpl., 53, 369 (1992)). In addition, tacrolimus has been used successfully in clinical organ transplantation, where it has been estimated to be three to ten orders of magnitude more potent than CsA in liver transplantation.

While CsA and tacrolimus have both demonstrated an immunosuppressive effect on the prevention of acute allograft rejection, recent experimental and clinical trials have shown that CsA and tacrolimus have a different immunosuppressive activity with respect to the rescue of ongoing acute allograft rejection (Ochiai et al., Transpl. Proc., 44, 729 (1987); Cianco et al., Transpl. Proc., 27, 812 (1995); Japanese tacrolimus Study Group, Transpl. Proc., 23, 3071 (1991); Laskow et al., Transpl. Proc., 27, 809 (1995); Murase et al., Transpl., 50, 186 (1990)). Ongoing rejection is considered as phases or stages of acute rejection or late phase of acute rejection evidenced by significant lymphocyte local infiltration and graft destruction. It occurs when an animal or human patient is administered an insufficient amount of immunosuppressant drugs, resulting in insufficient immunosuppression and the development of graft rejection.

Interestingly, it has been found that optimal doses of tacrolimus administered several days after transplantation (i.e., after allograft rejection has begun) can significantly increase allograft survival. In contrast, optimal doses of CsA administered after allograft rejection has no effect on graft survival. Thus, tacrolimus can be used not only as a prophylactic treatment to prevent acute allograft rejection, but also as an anti-rejection treatment to rescue a graft from ongoing rejection. In contrast, CsA has demonstrated its use as a prophylactic treatment only.

While graft survival rates have significantly improved, the incidence of acute allograft rejection with CsA, for example, remains as high as 69%, in part due to insufficient immunosuppression as a result of misdosage (Tarantino et al., Transpl., 52, 53 (1991)). Furthermore, the effectiveness of immunosuppressive drugs, such as CsA and tacrolimus, is limited by adverse effects that are generally concentration related and often prohibit maximum immunosuppression. For example, CsA can cause hypertension, hyperlipidemia, tremor and hirsutism, as well as profound and irreversible nephrotoxicity, hepatoxicity and cardiotoxicity (McEvoy, et al., Am. Soc. Hosp. Pharm., 2476-2478 (1994); Wiederrecht et al., Ann. NY Acad. Sci., 696, 9-19 (1993); Faulds et al., Drugs, 45, 953-1040 (1996)). Similarly, tacrolimus has been shown to cause nephrotoxicity, neurotoxicity and altered glucose metabolism (Yokota et al., Transpl. Proc., 21, 1066 (1989); Ochiai et al., Transpl., 44, 729 (1987); Shapiro et al., Transpl. Proc., 19, 57 (1987)). In addition, misdosage can occur as a result of low or variable absorption of immunosuppressive drugs. For example, the absorption of CsA is reported to be approximately 30% with high intrapatient and interpatient variability and bioavailability depending on gastric motility, mucosal integrity, presence of food and bile flow (Liao et al., Pharmacotherapy, 16, 401-408 (1996)). As a consequence of misdosing, toxicity and/or low absorption as well as a given individual's sensitivity to a particular immunosuppressant, ongoing acute allograft rejection due to insufficient immunosuppression remains a problem in the area of tissue and organ transplantation and only highlights the complexity of the acute rejection process.

Thus, there exists a need for an immunosuppressant treatment method that will suppress ongoing allograft rejection, such as that which occurs as a result of improper dosage or low absorption. Furthermore, there exists a need for an immunosuppressant treatment method that will suppress ongoing allograft rejection without requiring toxic levels of immunosuppressive drugs.

In view of the above, it is an object of the present invention to provide a method of suppressing ongoing acute allograft rejection, such as that which results from an insufficient immunosuppression. This and other objects and advantages, as well as additional inventive features, of the present invention will become apparent to one of ordinary skill in the art upon reading the detailed description provided herein.

### BRIEF SUMMARY OF THE PRESENT INVENTION

The present invention provides a method of suppressing ongoing acute allograft rejection. In one embodiment, the method comprises administering to a host experiencing ongoing acute allograft rejection an IL10 inhibitor and an IL2 inhibitor (or tacrolimus alone) in amounts effective to rescue the allograft from ongoing acute rejection. In another embodiment, the method comprises administering to a host experiencing ongoing acute allograft rejection, which is due to insufficient immunosuppression by an IL2 inhibitor, an IL10 inhibitor in an amount effective to rescue the allograft from ongoing acute rejection.

### DETAILED DESCRIPTION OF THE PRESENT INVENTION

The present invention provides methods for suppressing ongoing acute allograft rejection. The present invention is premised on the surprising discovery that an IL10 inhibitor and an IL2 inhibitor can suppress ongoing allograft rejection.

In view of the above, the present invention provides, in one embodiment, a method of suppressing ongoing acute allograft rejection in a host by administering an effective amount of an IL10 inhibitor and an effective amount of an IL2 inhibitor to rescue the graft from ongoing acute rejection. Ongoing acute rejection due to insufficient immunosuppression can result from the limited immunosuppressive capacity and the potential mechanism of action of an immunosuppressive drug. Acute rejection starts at day 3-5 following transplantation in animal transplantation models and can be characterized, for example, by an elevation of lymphocyte infiltration and associated cytokine/cytotoxic factor production and evidence of graft destruction. It can be identified by biopsy.

The host that can be treated in accordance with the method of suppressing ongoing acute allograft rejection can be any mammal, including a human. The allograft can be any tissue or organ. The term “tissue” includes individual cells, such as blood cells, including progenitors and precursors thereof. Examples of tissues include cornea, bone marrow and pancreatic island. Examples of organs that may be rescued from ongoing acute rejection include heart, kidney, liver, lung, pancreas, bladder, small intestine, skin and others.

The IL10 inhibitor used to rescue the graft from ongoing acute rejection can be any suitable inhibitor, whether it acts at the level of DNA, RNA or protein and affects IL10 or its receptor directly or indirectly. An example of an IL10 inhibitor is an antagonist, such as an anti-IL10 antibody. An antibody can be raised to the IL10 cytokine protein, an analog or an immunogenic fragment thereof, both in its naturally occurring form and its recombinant form. Additionally, antibodies can be raised to IL10 in either an active form or an inactive form, the difference being that antibodies to the active cytokine are more likely to recognize epitopes which are only present in the active conformation. Further, the IL10 protein, analog or fragment can be joined to other materials, such as polypeptides, through covalent bonding or noncovalent interaction, for example.

An anti-IL10 antibody can be a polyclonal or a monoclonal antibody. Preferably, such an antibody is a monoclonal antibody. Anti-IL10 antibodies can be purchased from a commercially available source or can be prepared and characterized using methods well-known in the art (See, e.g., Howell et al., Antibodies, A Laboratory Manual (Cold Spring Harbor Laboratory 1988)). Briefly, a polyclonal antibody can be prepared by repeated immunizations of an animal with an IL10 immunogen and collecting antisera from the immunized animal. A wide range of animal species can be immunized for the production of antibodies as is known in the art. Such animal species include, but are not limited to, goat, rabbit, mouse, rat, hamster, or guinea pig. Preferably, a goat or rabbit is used for the production of polyclonal antibodies due to the relatively large blood volume of such animals. A monoclonal antibody can be obtained by immunizing an animal with an IL10 immunogen, removing spleen or lymph cells from the animal, and fusing the spleen or lymph cells with plasmacytoma cells using a standard fusion protocol to produce antibody-secreting hybridomas.

Alternatively, the IL10 inhibitor can be an IL10 antagonist that can bind to the IL10 receptor and inhibit ligand binding to the receptor and/or inhibit the ability of IL10 to elicit a biological response. Such IL10 antagonists include antibodies to the IL10 receptor, mutant IL10 ligands that bind to IL10 receptors without effect, and the like. Also, the IL10 inhibitor can be a molecule that blocks the upstream or downstream signals of IL10. Alternatively, an IL10 inhibitor can be an antisense molecule (see, e.g., WO 97/31532, Senior, Biotech. Genet. Eng. Rev. 15: 79-119 (1998); Bird et al., Biotech. Genet. Eng. Rev. 9: 207-227 (1991); Matzke et al., Trends Genet. 11(1): 1-3 (1995); Baulcombe, Plant Mol. Biol. 32(1-2): 79-88 (1996); Castanatto et al., Crit. Rev. Eukaryot. Gene Exp. 2(4): 331-357 (1992); and Rossi, Trends Biotechnol. 13(8): 301-306 (1995)) or a ribozyme specific for the IL10 mRNA transcript or a DNA binding protein that inhibits or prevents expression of IL10. The antisense therapeutic nucleic acid itself can further comprise a ribozyme sequence.

Antisense nucleic acid molecules can be generated in accordance with methods known in the art. The nucleic acid molecule introduced in antisense inhibition generally is substantially identical to at least a portion, preferably at least about 20 continuous nucleotides, of the nucleic acid to be inhibited, but need not be identical. The antisense nucleic acid molecule can be designed such that the inhibitory effect applies to other proteins within a family of genes exhibiting homology or substantial homology to the nucleic acid. The introduced antisense nucleic acid molecule also need not be full-length relative to either the primary transcription product or fully processed mRNA. Generally, higher homology can be used to compensate for the use of a shorter sequence. Furthermore, the introduced sequence need not have the same intron or exon pattern, and homology of non-coding segments will be equally effective.

Ribozymes can be designed that specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature 334: 585-591 (1988). Preferably, the ribozyme comprises at least about 20 continuous nucleotides complementary to the target sequence on each side of the active site of the ribozyme.

As used herein, an effective amount of an IL10 inhibitor means an amount sufficient to rescue the graft from ongoing acute rejection. The effective amount of an IL10 inhibitor for a particular host may vary depending on such factors as the IL10 inhibitor used, the overall health of the host, the toxicity of the Il-10 inhibitor, and the route of administration, among others. The effective amount of an IL10 inhibitor can be determined using a transplantation assay, such as the one described in Example 1. Furthermore, other assays by which to determine an effective amount of an IL10 inhibitor are known in the art.

Generally, the IL10 inhibitor is administered as a pharmaceutical composition comprising an effective amount of an IL10 inhibitor and a pharmaceutically acceptable carrier. A pharmaceutically acceptable carrier can be any compatible non-toxic substance suitable for delivering the compositions of the invention to the host. Examples of such carriers include normal saline, Ringer's solution, dextrose solution, and Hank's solution. Nonaqueous carriers, such as fixed oils and ethyl oleate, can also be used. A preferred carrier is 5% dextrose/saline. The carrier can contain minor amounts of additives which enhance the isotonicity and the chemical stability of the pharmaceutical composition, such as buffers and preservatives. The requirements for pharmaceutically acceptable carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986)).

Preferably, administration is begun as soon as ongoing rejection is identified, such as by one or more functional parameters, e.g., blood parameters, or biopsy. The IL10 inhibitor can be administered orally, parenterally, or by any other suitable means. Oral administration can be carried out using well-known formulations which protect the compound from gastrointestinal protease. Preferably, the IL10 inhibitor is administered parenterally by intraperitoneal, intravenous, subcutaneous or intramuscular injection of the composition or by any other acceptable systemic method. Also, the IL10 inhibitor can be delivered by an implantable drug delivery system.

The method further comprises the simultaneous administration of an inhibitor of IL2 in an amount effective to rescue the allograft from ongoing acute rejection. Alternatively, the IL10 inhibitor also can inhibit IL2. Any suitable inhibitor of IL2 can be used. The IL2 inhibitor can act at the level of DNA, RNA or protein and can affect IL2 or its receptor directly or indirectly. An example of an IL2 inhibitor is an antagonist, such as an anti-IL2 antibody (see discussion above with respect to anti-IL10 antibody). Alternatively, the IL2 inhibitor can be an IL2 antagonist that can bind to the IL2 receptor and inhibit ligand binding to the receptor and/or inhibit the ability of IL2 to elicit a biological response (see discussion above with respect to IL10 antagonist). The IL2 inhibitor also can be a molecule that blocks the upstream or downstream signals of IL2, such as rapamycin and the like. Alternatively, the IL2 inhibitor can be an antisense molecule or a ribozyme specific for the IL2 mRNA transcript or a DNA binding protein that inhibits or prevents expression of IL2. Other examples of IL2 inhibitors include CsA, which inhibits IL2 mRNA transcription, cycloheximide, a p38 MAP kinase inhibitor, and a negatively acting transcription factor, such as negative regulatory element-A (NREA). CsA is a preferred IL2 inhibitor.

An effective amount of an IL2 mRNA inhibitor and the determination of an effective amount are as described above with respect to an IL10 inhibitor. Pharmaceutical compositions comprising an effective amount of an IL2 inhibitor and a pharmaceutically acceptable carrier are also as described above with respect to an IL10 inhibitor. In this regard, the IL10 inhibitor and the IL2 inhibitor can be administered in the same or different compositions and by the same or different routes. In any event, if the IL10 inhibitor and the IL2 inhibitor are administered by different routes, they must be administered such that the benefit of co-administration is realized. If ongoing allograft rejection occurs during administration of an IL2 inhibitor, such as an IL2 mRNA transcription inhibitor, preferably the IL10 inhibitor is administered at an early stage of acute rejection.

In another embodiment, the present invention provides a method of suppressing acute allograft rejection due to insufficient immunosuppression by an IL2 inhibitor. The method comprises administering to a host experiencing ongoing acute allograft rejection an IL2 inhibitor in an amount effective to rescue the allograft from ongoing acute rejection. The host, allograft and tissue are as described above. The IL10 inhibitor also is as described above as is an effective amount of an IL10 inhibitor. Pharmaceutical compositions for administration of an IL10 inhibitor, routes of administration and timing of administration also are as described above.

### EXAMPLES

The present invention is described in the following examples. These examples serve to illustrate further the present invention and are not intended to limit the scope of the invention.

### Example 1

This example demonstrates the determination of the optimal doses of CsA and tacrolimus used as primary therapy to prevent acute allograft rejection in rat heart transplantation.

Inbred male Lewis rats (LEW) (RT1¹), weighing 200-250 g, were used as heart transplantation recipients. Inbred male LEW and inbred male Brown Norway (BN) (RT1^{a}), weighing 150-200 g, were used as isograft and allograft donor rats, respectively. Heart transplantation was performed using the modified technique of Ono and Lindsey (Ono et al., J. Thorac. Cardiovasc. Surg., 57, 225 (1969)) with the donor aorta and pulmonary artery anastomosed, end to side, to the recipient's abdominal aorta and inferior vena cava, respectively. Transplantation recipients received either CsA or tacrolimus from day 0 to day 10 after transplantation and were monitored daily for rejection. Rejection of heart grafts was defined as the complete cessation of impulse and was confirmed visually after laparatomy. Various doses (3.2, 10, or 32 mg/kg) of CsA (Fujisawa Pharmaceutical Co., Ltd., Osaka, Japan, Lot No. 060975L) were dissolved in 5 ml of pure olive oil and were administered orally to recipient rats (five rats per dose) daily from day 0 to day 10 after transplantation. Various doses (1, 3.2, or 10 mg/kg) of tacrolimus (Fujisawa, Lot No. 701732) were dissolved in 5 ml of water and administered orally to recipient rats (five rats per dose) daily from day 0 to day 10 after transplantation. Control isograft and allograft recipients (5 rats each) received no immunosuppressant drugs after transplantation. Control heart isografts survived a median survival time (MST) of >100 days. In contrast, control heart allografts survived an average of 6 days. The heart allografts of allograft recipients receiving 3.2/kg/day of CsA survived an MST of 11 days. The allografts of those receiving 10 mg/kg/day of CsA survived an MST of 21 days. The allografts of those receiving 32 mg/kg/day of CsA survived an MST of 20 days. Based on these results, the optimal dose of CsA was determined to be 10 mg/kg/day. The heart allografts of those receiving 1.0 mg/kg/day of tacrolimus survived an MST of 12 days. The allografts of those receiving 3.2 mg/kg/day of tacrolimus survived an MST of 19 days and those receiving 10 mg/kg/day survived an MST of 21 days. The optimal dose of tacrolimus was determined to be 3.2 mg/kg/day.

### Example 2

This example demonstrates that tacrolimus but not CsA, can rescue ongoing heart allograft rejection.

The failure to administer immunosuppressant drugs for the first four days following allograft heart transplantation results in ongoing graft rejection as determined by local lymphocyte infiltrations and their related cytokine/cytotoxic gene expression (Fujimura et al., Transplant Proc. 30:1023 (1998)). Therefore, drug administration was halted from day 0-day 4 after heart transplantation in order to initiate the transplantation process. The transplantation methods described in Example 1 were used, except that optimal doses of CsA and tacrolimus were administered daily to graft recipients starting from day 5 after grafting and continuing for 10 days (i.e., days 5-14 post-transplantation). In the absence of CsA or tacrolimus, control isografts survived an MST of >30 and control allografts survived an MST of 6 days. The daily administration of 3.2 mg/kg/day of tacrolimus given on days 5-14 post-transplantation resulted in an MST of 19 days, thereby extending the survival of the graft about 13 days. In contrast, the daily administration of 10 mg/kg/day of CsA resulted in an MST of only 7 days. These results show that tacrolimus, but not CsA, can rescue heart allografts from ongoing graft rejection.

### Example 3

This example demonstrates that tacrolimus, but not CsA, can rescue ongoing renal allograft rejection.

To examine whether tacrolimus or CsA could prevent ongoing renal graft rejection, renal grafts were transplanted from male BN rats weighing 150-250 g (RT1ⁿ RT2^{a}, Seiwa, Japan) to male LEW rats weighing 250-350 g (RT1¹ RT2^{b}, Charles River, Japan). Male LEW rats weighing 250-350 g and male BN rats weighing 150-250 g were kept under specific pathogen-free conditions in the animal facility at Fujisawa Pharmaceutical Co., Ltd., Osaka, Japan. Renal transplants were performed using the modified technique described by Fisher and Lee (Fisher et al., Surgery, 58, 904 (1965)). Kidneys were transplanted end-to-side anastomoses of the renal artery and vein with patches of aorta and inferior vena cava, respectively. The donor ureter was directly implanted into the recipient bladder over a fine polyethylene internal stent. All recipients were bilaterally nephrectomized at the time of transplantation. The graft ischemic time was approximately 30 minutes. Survival of the kidney transplant was measured as time of rat survival. Graft rejection was confirmed by histological examination of kidney tissue from recipients with the observation of interstitial hemorrhage, tubular necrosis, venous endotheliatitis and diffuse lymphoplasmacytic infiltration indicating graft rejection. Various doses (3.2, 10, and 32 mg/kg) of CsA (Fujisawa, Lot No. 060975L) were dissolved in 5 ml of pure olive oil and administered orally to recipient rats (seven rats per dose) daily for 13 days starting from day 2 or day 4 after transplantation. Various doses (0.32, 1.0, 3.2 mg/kg) of tacrolimus (Fujisawa, Lot No. 701732) were dissolved in 5 ml of water and administered orally to recipient rats (seven rats per dose) daily for 13 days starting from day 2 or day 4 after transplantation. Control isograft and allograft recipients received either distilled water, olive oil, or placebo after transplantation. Renal isografts were not rejected and survived for more than 100 days. Control renal allografts were rejected at an MST of 8 days for water, 8 days for olive oil, and 9 days for placebo. Untreated renal allograft controls were rejected at day 8. However, all of 5 recipient rats treated with 3.2 mg/kg/day tacrolimus from day 2-14 after grafting and 3 of 5 recipients treated from day 4-16 after grafting survived more than 50 days. In contrast, recipient rats treated with 10 mg/kg/day CsA from day 2-14 after grafting survived 19 days and those treated from day 4-16 after grafting survived 13 days. These results show that tacrolimus but not CsA can significantly overcome ongoing renal allograft rejection in the rat.

### Example 4

This example demonstrates that tacrolimus, but not CsA, can dramatically inhibit CD8⁺ (cytotoxic T cell) and natural killer (NK) cell infiltration into heart allograft tissue during anti-rejection therapy to rescue ongoing heart allograft rejection.

Using the methods described in Example 1, optimal doses of CsA (10 mg/kg/day) and tacrolimus (3.2 mg/kg/day) were administered orally to recipient rats from day 5 for 10 days after heart transplantation. The presence of CD4+, CD8+, NKR-P1A, and ED2 positive cells was examined on day 5, 7 and 10 after transplantation by immunohistological examination of graft tissue immunostained with specific antibodies to various immune cell populations. For immunohistology, 4 μm frozen sections of heart tissue were cut, fixed with acetone and exposed to monoclonal antibodies (Pharmingen, San Diego, Calif.) against markers for CD4+ (helper T cells), CD8⁺ (cytotoxic T cells), NKR-P1A (natural killer cells), and ED2 (macrophage cells) positive cells. Then the sections were incubated with peroxidase-conjugated anti-mouse IgG antibodies. The bound peroxidase was detected using 3-amino-9-ethylcarbazole. The results were scored quantitatively. In each tissue section, three random representative fields were counted by visual microscope (×40) to determine the average cell number per tissue. Cell populations which stained positively in the heart tissues of the allograft control progressively increased up to day 10 for CD4+, CD8+, NKR-P1A, and ED2 positive cells. Tacrolimus and CsA showed no significant suppression of all of the cell infiltration into the local graft tissues at day 7. However, both tacrolimus and CsA showed comparable activity in partially blocking the CD4⁺ and ED2 cell filtration at day 10. In contrast, tacrolimus, but not CsA, significantly blocked CD8⁺ and NKR-P1A cell infiltration at day 10. These results demonstrate that tacrolimus, but not CsA, blocks the infiltration of cytotoxic T cells and natural killer cells, suggesting that tacrolimus may rescue heart allografts from ongoing rejection by blocking the infiltration of cytotoxic T cells and natural killer cells to the graft tissue.

### Example 5

This example demonstrates that tacrolimus down-regulates the expression of IL10 mRNA in allograft heart recipients during anti-rejection therapy to rescue ongoing heart allograft rejection.

Using the methods described in Example 1, either no drug, 10 mg/kg/day CsA, or 3.2 mg/kg/day tacrolimus were administered orally to recipient rats at day 5 after transplantation. At day 5, 7 and 10 after transplantation, total RNA was extracted from heart graft tissues and tested by reverse transcriptase-polymerase chain reaction (RT-PCR) for cytokine mRNAs. Total RNA was extracted from transplanted heart tissue by TRIZOL (GIBCO BRL, Gaithersburg, Md.). RT-PCR was performed by reverse transcribing total RNA into cDNA using random hexamers (GIBCO BRL, Gaithersburg, Md.). The reverse-transcribed material was added to the PCR mixture with sense and anti-sense primers of IFNγ, IL2, IL4, IL10, IL1β, IL6, TNFα, TGFβ, Granzyme, Perforin or G3PDH and Taq DNA polymerase (Boehringer Mann heim GmbH, Mannheim, Germany). Amplification was performed with optimized cycles for each cytokine or G3PDH by ThermalCycler (Perkin-Elmer, Branchburg, N.J.). PCR product was applied to 2.3% agarose gel, resolved by electrophoresis, and visualized by ethidium bromide staining. The intensity of the gene-specific bands was obtained by the Gel Doc 1000 documentation system (BIO-RAD Laboratories, Hercules, Calif.). G3PDH mRNA was amplified as an internal control for RT-PCR. Compared with isograft controls, allogenic controls showed that mRNAs for IL1β, IL2, IL6, IL10, IFNγ, TNFα, TGFβ, granzyme and perforin were significantly upregulated at day 5, decreasing thereafter in the allograft control when compared with the isograft. Both CsA and tacrolimus comparably inhibited IFNγ, IL2, IL1β, IL6, TNFα, TGFβ, Granzyme, and Perforin mRNA expression in a range of from about 20% to about 50% at days 7 and 10 compared with allograft control. However, at days 7 and 10, tacrolimus suppressed IL10 mRNA expression more than CsA. This example demonstrates that tacrolimus, but not CsA, can suppress the expression of IL10 mRNA in allograft heart recipients during anti-rejection therapy to rescue ongoing heart allograft rejection.

### Example 6

This example demonstrates that tacrolimus down-regulates the expression of IL10 protein in allograft heart recipients during anti-rejection therapy to rescue ongoing heart allograft rejection.

The transplantation method described in Example 5 was used. Optimal doses of tacrolimus or CsA were administered at day 5 post-transplantation. The levels of IL2, IL4 and IL10 proteins in serum peripheral lymphocytes were measured at day 7 post-transplantation by ELISA assay. CsA and tacrolimus suppressed the expression of IL2 and IL4 protein equally. In contrast, tacrolimus suppressed IL10 protein expression 73%, compared with the allograft control, whereas CsA did not suppress IL10 protein compared with the allograft control. These results show that tacrolimus, but not CsA, down-regulates the level of IL10 in allograft heart recipients during anti-rejection therapy to rescue ongoing heart allograft rejection.

### Example 7

This example demonstrates that an inhibitor of IL10, such as an anti-IL10 antibody, in combination with CsA rescues a heart allograft from ongoing acute allograft rejection.

The rats and transplantation methods described in Example 2 were used. Anti-mouse IL10 antibody (R&D System, Cat. No. AF-417-NA, Lot No. NU07) was suspended in normal saline. Heart allograft recipients received no drug for days 0-4 post-transplantation to allow the occurrence of acute graft rejection. At day 5, heart allograft recipients were given either no drug (control), 10 mg of CsA alone administered for 10 days (i.e., days 5-14 post-transplantation), 1 mg of anti-IL10 antibody for 2 days (days 5-6 post-transplantation), or 10 mg CsA for 10 days and 1 mg of anti-IL10 antibody for 2 days. MST of the isograft control (no drugs) was more than 30 days, while MST of the allograft control was 6 days. The MST of the recipient allograft treated with either CsA or anti-IL10 alone was 7 days (i.e., similar to allograft control). In contrast, the average survival of the recipient allograft treated with CsA and anti-IL10 was 13 days. These results show that the ongoing rejection of a heart allograft can be rescued by the administration of an inhibitor of IL10, such as anti-IL10 antibody and CsA at day 5 post-transplantation. Further, the results indicate that CsA alone does not inhibit ongoing allograft rejection due to insufficient suppression of IL10.

All of the references cited herein, including patents, patent applications, literature publications, and the like, are hereby incorporated in their entireties by reference.

While this invention has been described with an emphasis upon preferred embodiments, it will be obvious to those of ordinary skill in the art that variations of the methods can be used and that it is intended that the invention can be practiced otherwise than as specifically described herein. Accordingly, this invention includes all modifications encompassed within the spirit and scope of the invention as defined by the following claims.

1. A method of suppressing ongoing acute allograft rejection, which method comprises administering to a host experiencing ongoing acute allograft rejection an interleukin-10 (IL10) inhibitor and an interleukin-2 (IL2) inhibitor in amounts effective to rescue the allograft from ongoing acute rejection.

2. The method of claim 1, wherein said IL10 inhibitor is an IL10 antagonist.

3. The method of claim 2, wherein said IL10 antagonist is an anti-IL10 antibody.

4. The method of claim 2, wherein said IL10 antagonist is an IL10 receptor antagonist.

5. The method of claim 4, wherein said IL10 receptor antagonist is an anti-IL10 receptor antibody.

6. The method of claim 1, wherein said IL10 inhibitor is a compound which blocks the upstream or downstream signals of IL10.

7. The method of claim 1, wherein said IL10 inhibitor and said IL2 inhibitor are administered during acute rejection.

8. The method of claim 1, wherein said allograft is selected from the group consisting of heart, kidney, liver, lung, pancreas, bladder, small intestine, and skin.

9. The method of claim 1, wherein the inhibitor of IL2 is an inhibitor of IL2 mRNA transcription.

10. The method of claim 9, wherein said inhibitor of IL2 mRNA transcription is cyclosporin A (CsA).

11. A method of suppressing ongoing acute allograft rejection, which method comprises administering to a host experiencing ongoing acute allograft rejection, which is due to insufficient immunosuppression by an IL2 inhibitor, an IL10 inhibitor in an amount effective to rescue the allograft from ongoing acute rejection.
